(19) United States
(12) Patent Application Publication
(10) Pub. No.: US 2010/0105633 A1
(43) Pub. Date: Apr. 29, 2010

(54) PROCESS

(75) Inventors: Kjell Morton Varum, Porsgrunn (NO); Kurt Ingar Draget, Porsgrunn (NO); Olav Smidsrod, Porsgrunn (NO)

Correspondence Address: BACON & THOMAS, PLLC, 625 SLATERS LANE, FOURTH FLOOR, ALEXANDRIA, VA, 22314-1176, US

(73) Assignee: Advanced Biopolymers AS, Porsgrunn (NO)

(21) Appl. No.: 12/448,466

(22) PCT Filed: Dec. 20, 2007
(86) PCT No.: PCT/GB2007/004899

§ 371 Date: Nov. 17, 2009

(30) Foreign Application Priority Data
Dec. 22, 2006 (GB) 0625813.1

(51) Int. Cl. A61K 31/722; C08B 37/08; A61P 43/00

(52) U.S. Cl. 514/55; 536/20

(57) ABSTRACT

The invention relates to a process for the preparation of water-soluble chitosan salts, to chitosan products produced by this process and to their uses, e.g. in pharmaceutical or veterinary compositions or as a food or cosmetic additive. More specifically, the invention relates to a process for preparing a water-soluble chitosan salt which comprises: (a) contacting a solid chitosan with at least one protic acid whereby to produce a water-soluble chitosan salt; and (b) optionally recovering said salt, wherein said chitosan is converted into said salt whilst remaining substantially in the solid state. Gaseous or liquid protic acids may be used, including hydrochloric, acetic and propionic acids.

This invention relates to a process for the preparation of water-soluble chitosan salts, to chitosan products produced by this process and their uses.

More particularly, the invention relates to a process for producing water-soluble chitosan salts performed under conditions in which a solid chitosan is converted into salt form whilst substantially retaining its original solid state form (i.e. it does not dissolve during the process). Such a process avoids the need for any costly recovery step to obtain the desired chitosan salt.

Chitin is a naturally occurring nitrogenous mucopolysaccharide of formula (C₈H₁₃N₅)ₙ which is found in the exoskeletons of insects, crustaceans (e.g. shrimp, crab, prawn and lobster) and invertebrates in general. More particularly, chitin is poly N-acetyl-D-glucosamine. Thus chitin consists of (1→4)-linked 2-acetamido-2-deoxy-3-D-glucose (GlcNac; the A-unit). The physical structure of chitin is highly ordered, and the most abundant form is α-chitin which is available as a waste material from the shellfish food industry. In α-chitin the chains are antiparallel and extensively hydrogen-bonded. Another form is β-chitin, which can be isolated from, for example, the pen of the squid Loligo and the spines of the diatom Thalassiosira fluviatilis. In β-chitin the chains are parallel and the chains are less hydrogen-bonded compared with α-chitin.

Chitin is insoluble in water, even at acidic pH-values, and in most organic solvents. This has served to limit the applications for which it can be used.

The N-acetyl groups in chitin can be cleaved off to yield N-deacetylated chitin or chitosan. Chitosan may be regarded as a family of polysaccharides consisting of (1→4)-linked A-units and units of 2-amino-2-deoxy-β-D-glucose (GlcN; the D-unit) in varying relative abundances and sequences. In contrast to chitin, chitosan is soluble in various acidic solvents. Therefore, chitosan has received much more attention as a biopolymer for a wide range of applications such as in pharmaceutical and cosmetic compositions, as fillers, absorbants, carriers and supports.

When chitosan is prepared from chitin, the final step in the procedure invariably involves treatment with a strong alkali, e.g. sodium hydroxide, which is effective to remove the acetyl groups. As a result of this treatment, most commercially available chitosan is produced and sold in a form in which very few of the amino groups are protonated (this is generally referred to as the ‘free amine’ form of chitosan). This particular form of chitosan is generally insoluble in aqueous solvents, e.g. in water.

In order to dissolve chitosan in aqueous-based systems, the chitosan must be Made more hydrophilic (i.e. converted to a polyelectrolyte). Typically this is achieved by adding a controlled amount of an acid, e.g. 1% acetic acid, which results in protonation of the free amino groups of the D-units. Partial disruption of the hydrogen bonds within the chitosan structure, believed to be caused by the introduction of positive charges along the polymer molecule, leads to eventual dissolution. This is similar to processes performed with other polysaccharides, such as alginic acid/alginate. This polysaccharide is also insoluble in water when it is prepared as alginic acid (similar to the free amine form of chitosan), but is soluble in water when it is converted to the hydrophilic alginate form (similar to chitosan in the salt form).

A smaller quantity of commercially produced chitosan is converted into different salt forms, i.e. with a protonated amino-group in the D-units and a negatively charged counter ion (e.g. acetate, chloride or glutamate), which make it soluble in water without the need for the addition of an acid. In general, such water-soluble chitosan is prepared by making a slurry of the chitosan in water and then adding stoichiometric amounts of acid to the slurry in order to obtain a chitosan solution. A disadvantage of this method is the requirement for large amounts of water in order to reduce the viscosity of the solution. This water must be removed by drying techniques such as freeze-drying, spray-drying, etc. in order to recover the chitosan salt in dry form. Such drying methods are expensive and therefore ineffective for a commercially viable process. When preparing highly pure chitosan there is a further disadvantage with this method of salt formation which is that any impurities present in the process water inevitably end up, after drying, as impurities in the final chitosan product. These impurities then have to be removed in a further post-treatment step, e.g. by washing (typically with organic liquids), or by means of a chromatographic procedure.

An alternative method for the preparation of dry, water-soluble chitosan salts involves precipitation of salts with inorganic acids in the presence of an alcohol or other organic liquid. For example, U.S. Pat. No. 2,201,762 teaches the precipitation of the sulfamic acid salt of chitosan from an aqueous solution by the addition of methanol. However, this method is generally very time-consuming in order to achieve complete conversion to the desired salt. Also, the chitosan product still has to be purified by washing with organic liquids before this can be dried. This is costly and therefore commercially non-viable.

In U.S. Pat. No. 6,326,475 a method is described for the production of an acid-chitosan complex in which a chitosan is vigorously mixed with an acid and an effective amount of water. In this method, water is added as a carrier for the acid, allowing it to dissolve, ionise and penetrate the chitosan particles. This earlier patent teaches that the addition of water is essential to impart mobility to the acid molecules such that at least partial protonation of the chitosan can be achieved in order to produce a water-soluble acid-chitosan combination. The acid-chitosan material is formed as a homogenous semi-solid mixture which must be dried and ground to produce a particulate product. Such additional processing steps render the process expensive and ineffective as a commercially viable method for the production of water-soluble chitosan materials. Large amounts of water have to be used in this method to produce readily and fully soluble chitosan products.

Therefore there still exists a need in the art for an alternative, e.g. a more efficient and effective, method of preparing water-soluble chitosan salts.

The Applicant has now developed a method for the production of water-soluble chitosan salts in which a solid chitosan product is effectively protonated whilst remaining substantially in the solid state, preferably in particulate or free-flowing form. A chitosan product may be considered to remain “substantially in the solid state” when less than 30% by weight, preferably less than 20% by weight, more preferably less than 10% by weight, e.g. less than 5% by weight, of the chitosan product changes state (i.e. from a solid to a swollen state (e.g. a gel) or to a liquid (e.g. dissolved)).

As described herein, the method according to the invention is preferably carried out by bringing the solid chitosan material into contact either with a liquid or gaseous protic acid. Where the acid used is provided in the form of a gas or vapour, it is preferred that the solid chitosan starting material should not significantly alter in state during its conversion to a water-soluble form, i.e. it preferably remains in solid form. Where the acid used is in the form of a liquid, it is preferred that the chitosan starting material should not dissolve to any significant degree in the liquid acid. For example, it is preferred that greater than 70% by weight, preferably greater than 90% by weight, especially preferably greater than 95% by weight of the original chitosan remains undissolved following reaction with the liquid acid. Regardless of the precise form of the acid used, where the original chitosan material is in particulate form, it is particularly preferred that this should retain essentially the same particle size during the process of conversion to the acid form.

The method of the invention may be carried out by contacting (e.g. physically mixing) a substantially dry, protic acid (e.g. a liquid or gaseous acid) and chitosan. Preferably, the reaction is carried out essentially in the absence of any additional water. The resulting product is a water-soluble chitosan salt or complex which remains in particulate (i.e. undissolved) form and which, if desired, can be readily (and thus cheaply) separated from any excess acid. According to this method, water-soluble chitosan can therefore be produced without any of the associated disadvantages of the prior art procedures. In particular, there is no need for the removal of large quantities of water or the need for any post-treatment step to improve the level of purity of the chitosan. Furthermore, since the Applicant's method does not necessitate the use of organic solvents, this further reduces the cost of the production process whilst at the same time making this more environmentally acceptable.

To date, it has generally been considered essential to impart mobility to the acid molecules and thereby accomplish protonation of chitosan by the addition of a large volume of water which acts as a carrier (see e.g. U.S. Pat. No. 6,326,475). However, the Applicant has found that the low level of residual water present in the chitosan particles is sufficient to effect protonation without the need for the further addition of water (referred to herein as “process water”). That protonation could be achieved essentially in the absence of any additional water is considered surprising in light of the teaching of the prior art.

Viewed from one aspect the invention thus provides a process for the preparation of a water-soluble chitosan salt which process comprises (e.g. consists essentially of) the following steps:
(a) contacting a solid chitosan with at least one protic acid whereby to produce a water-soluble chitosan salt; and
(b) optionally recovering said salt,
   wherein said chitosan is converted into said salt whilst remaining substantially in the solid state.

Preferably, the acid used in the process according to the invention will be substantially dry.

The process of the invention is preferably conducted essentially in the absence of any additional process water. In this respect, it is preferred that no process water is used. However, small amounts of process water may nevertheless be used whilst still achieving the desired effect of the invention. If process water is added, this is preferably in an amount of less than 10% by weight, more preferably less than 5% by weight, yet more preferably less than 2% by weight, e.g. less than 1% by weight (based on the total weight of the chitosan and the acid). As used herein, the term “process water” is intended to refer to additional water which may either be added to the chitosan or to the acid prior to performing the process herein described, or which may be added to the mixture of chitosan and acid during the reaction. The term “process water” is not intended to refer to any residual water in the chitosan starting material or which may inherently be present in the acid (e.g. liquid acid) used.

A distinguishing feature of the invention over the methods conventionally used in the art is that the chitosan is, and remains, in essentially solid (e.g. particulate) form. For example, in the case where the acid is a liquid acid, the chitosan remains substantially insoluble in the acid used. Similarly, where the acid used is a gaseous acid, the acid contacts the particulate chitosan without substantially altering the solid state of the chitosan. Thus, it is, possible to produce a solid, water-soluble chitosan salt (preferably a particulate, free-flowing chitosan salt) without the need to perform any time-consuming and costly methods for extraction of the product from unreacted acid. This is true regardless of whether a substantially dry, liquid acid or a gaseous acid is used.

The product produced by the process in accordance with the invention is a true salt in which at least partial protonation of the chitosan has occurred (i.e. chemical reaction between the two components has taken place). This is distinct from a physical blend or intimate mixture of an acid and a chitosan in which no protonation (or at least no significant degree of protonation) has occurred.

As used herein, a material is considered to be water-soluble when it substantially dissolves in excess water to form a solution, losing its particulate form and becomes essentially molecularly dispersed throughout the aqueous solution. More specifically, by “water-soluble chitosan” as used herein, is meant a chitosan that can be dissolved, preferably fully dissolved (i.e. more than 95% wt dissolved, e.g. more than 99% wt dissolved at a chitosan concentration of 1%) in an aqueous environment (e.g. when only water is added to the chitosan).

Preferably, the process of the invention is further carried out substantially in the absence of any organic solvent, such as for example an alcohol, e.g. methanol. Typically, the amount of any organic solvent (e.g. an alcohol) present will be less than 5% by weight, more preferably less than 1% by weight (based on the total weight of the chitosan and acid).

Chitosan is commercially available in a range of molecular weights and with varying degrees of deacetylation. Chitosans which may be used in the method of the invention may have a weight average molecular weight (M_{w}) within a very broad range, e.g. 1,000 to 5,000,000 g/mol. Preferably, however, M_{w} is 10,000 to 3,000,000 g/mol, especially 20,000 to 2,000,000 g/mol. Chitosans are commonly found to be deacetylated to a degree of 60 to 100%, e.g. 75 to 90%, and these are suitable for use as starting materials in the method of the invention. Highly deacetylated chitosans, for example those having an F_{A} of from 0 to 0.30, more preferably from 0 to 0.25, yet more preferably from 0.05 to 0.1, e.g. about 0.08, may be used in the invention (where F_{A} is the relative fraction of the saccharide units which are A rather than D units).

Alternatively, the chitosan for use in the invention may have a high degree of acetylation. For example, this may be a chitosan having an F_{A} of at least 0.25, preferably at least 0.3, more preferably up to 0.9, e.g. up to 0.7. Particularly preferred are chitosans which have an F_{A} in the range of from 0.25 to 0.80, especially 0.30 to 0.60, particularly 0.33 to 0.55, e.g. about 0.40 to 0.48. Such materials are described in WO 2004/069230 and WO 2004/068971 (to Advanced Biopolymers AS), the contents of which are incorporated herein by reference.

The chitosan used in the method of the invention is preferably produced using any of the processes described in WO 03/011912 (to Advanced Biopolymers AS), the entire content of which is incorporated herein by reference. For example, the chitosan may be produced by a process which involves swelling particulate chitin with an aqueous solution at a temperature below 30° C. (e.g. for a period of at least 36 hours and/or with the application of pressure or irradiation to accelerate the swelling). Such a process produces a swollen particulate chitin which may then be deacetylated by reaction with an alkaline solution at an elevated temperature. The chitosans produced by this process are distinguished by the nature of the oligomer products of their extended hydrolysis with concentrated acid; these show a pattern in size exclusion chromatography. Particularly preferred for use in the invention are chitosan materials having a degree of acetylation of 0.45 to 0.7 which on acid hydrolysis with 12M HCl for 9 hours at 40° C. yield a monomer and oligomer mixture which in size exclusion chromatography does not exhibit a pattern of progressively smaller peaks corresponding to monomers, dimers, trimers, tetramers, pentamers and hexamers.

The chitosan is preferably used in solid form, more preferably in particulate form, e.g. in the form of a powder, flakes or fibres. If necessary, this may be prepared by crushing, grinding or milling the solid chitosan material to produce particulates, e.g. having a particle size of from 1 to 5000 μm, preferably from 3 to 2000 μm. Since the rate of reaction between the chitosan and the acid may be dependent in part on the fineness of the particles, particles having a size of less than 1000 μm are particularly preferred.

The water content of the chitosan used as the starting material in the process herein described will typically be less than 30% by weight, preferably less than 25% by weight, e.g. less than 20% by weight, more preferably less than 18% by weight, especially preferably less than 15% by weight.

The process of the invention involves contacting chitosan with at least one acid as herein defined. Since in general the chitosan will be provided in the form of a solid, e.g. a free-flowing particulate, then in one embodiment this may be achieved by dispersing a finely divided suspension of the chitosan in a liquid acid. Although the chitosan may be added to the liquid acid essentially all at once, it will generally be more convenient to add this either continuously or batchwise. Although not essential; the resultant dispersion may be agitated to ensure effective mixing, e.g. by stirring. The reaction is allowed to continue until a sample removed from the reaction mixture is found to be soluble in water. Generally, the reaction time will be of the order of 0.5 to 200 hours, preferably 0.8 to 80 hours, e.g. about 24 hours. As the' reaction time is extended, the percentage yield of water-soluble chitosan increases. The reaction may conveniently be carried out at ambient temperature, i.e. at 20 to 25° C. However, the reaction mixture may be heated, for example to a temperature up to about 60° C. To ensure that the chitosan does not denature (and that any volatile acid used does not evaporate), the reaction mixture should be kept below 50° C.

Where the acid is a liquid, this may alternatively be sprayed onto the solid chitosan material, e.g. onto chitosan powder or flakes.

In another embodiment of the invention the chitosan may be contacted with a gaseous acid for a sufficient time and under suitable conditions to yield a soluble, partially or fully protonated chitosan salt. This process may be carried out at ambient pressure and temperature but is preferably carried out under vacuum, for example at a pressure of from 1 to 1000 mbar. The conditions may be chosen such that the acid used is partially or fully in the gaseous state. Such conditions may readily be determined by those skilled in the art for any given acid, but typically may be expected to involve a temperature of from 5° C. to 80° C. and a pressure of from 50 to 500 mbar, e.g. about 100 mbar.

When using a gaseous acid to perform the method of the invention, a particular advantage of this embodiment is the ease of recovery of the chitosan salt, i.e. there is no need for further separation and/or drying steps.

Although acids which are gaseous under ambient temperature and, pressure conditions and which are substantially dry are preferred for use in the method of the invention, it is also contemplated that water-stabilised acids or aqueous acid solutions that may be mobilised into the gas phase (e.g. by heating or under conditions of low pressure) may also be used. A particularly preferred acid for use in this regard is hydrochloric acid (HCl), which may be readily mobilised into the gas phase from an aqueous solution, for example from a 30 to 40% aqueous solution.

The relative proportion of chitosan and acid employed will depend in part on the nature of the acid used (e.g. whether this is mono- or polyprotic) and the desired degree of protonation of the final material. Where the acid is a liquid, then typically the particulate chitosan will be dispersed in about 0.5 to about 20 parts by weight, preferably about 0.8 to about 10 parts by weight, of acid.

Acids for use in the invention are protic acids which on dissociation provide at least one proton (H⁺). Both mono and polyprotic (e.g. diprotic) acids may be used. However, in general, monoprotic acids are preferred. The term “monoprotic” is intended to refer to an acid having one displaceable hydrogen atom per molecule. The term “polyprotic” should be construed accordingly.

A preferred aspect of the invention is that the acid used should be substantially dry, especially where the acid is a liquid acid. By “substantially dry” is meant that the acid (e.g. liquid acid) should have a water content of less than 15% by weight, preferably less than 10% by weight, preferably less than 5% by weight, more preferably less than. 3% by weight, yet more preferably less than 2% by weight, e.g. less than about 1% by weight. Acids which are substantially water-free, preferably anhydrous, are most preferred for use in the invention. Certain commercially available acids (e.g. hydrochloric acid) contain 1 to 50% water for stabilisation. Although such acids are considered to be concentrated, it may be necessary to further concentrate these prior to use in the method of the invention. This may be achieved by techniques known in the art such as distillation and evaporation.

Acids which may be used in the invention include not only those which are liquid at ambient temperature and pressure, but those which can be liquefied, i.e. which are solid or gaseous at ambient temperature. Acids which are liquid or gaseous under conditions of low pressure (e.g. below ambient pressure) and at ambient temperature may also be used. Acids which are liquid or gaseous under conditions of ambient temperature and pressure are, however, particularly preferred.

Most particularly preferred for use in the invention are fluid acids. By “fluid acid” is meant an acid that continually deforms under an applied shear stress regardless of the magnitude of the stress, e.g. an acid which is the form of a liquid, gas, vapour or plasma. Especially preferred are acids which are liquid under normal temperature and pressure conditions. Solid acids are less preferred, particularly those which require the addition of large amounts of water to facilitate complex formation with the chitosan.

The choice of acid for use in the invention will depend, at least in part, on cost, availability and toxicity. The requirement for low toxicity is particularly important in cases where the chitosan material is to be used in food or pharmaceuticals.

Both organic and inorganic acids may be used in the method of the invention. However, organic acids are generally preferred.

Examples of suitable inorganic acids include the halogen acids (i.e. HF, HCl, HBr and HI), sulphuric acid, nitric acid and phosphoric acid. Of these, hydrochloric and phosphoric acids are preferred. Most preferred is HCl.

Preferred acids are organic acids such as carboxylic acids, most preferably saturated carboxylic acids. Mono or polycarboxylic (e.g. dicarboxylic) acids may be employed, however monocarboxylic acids are generally preferred. Lower (e.g. C₁₋₆) alkanoic acids, preferably C₁₋₄ alkanoic acids are yet more preferred. Although in general these will be unsubstituted, these may optionally be substituted by one or more groups selected from halogen atoms (e.g. fluorine, chlorine, bromine), keto groups and hydroxy groups.

Examples of suitable polycarboxylic acids include citric, fumaric, malic, maleic, tartaric, oxalic, succinic and glutaric acids.

Examples of suitable monocarboxylic acids include formic, acetic, trifluoroacetic, propionic, butyric, isobutyric, pyruvic, glycolic, lactic, chloroacetic and dichloroacetic acids. Of these, formic, acetic, trifluoroacetic and propionic acids are preferred. Particularly preferred for use in the invention is acetic acid, most preferably substantially pure or glacial acetic acid. Glacial acetic acid having a purity level greater than 99.0%, e.g. about 99.5%, is particularly preferred.

Zwitterionic acids having at least one displaceable proton may also be used in the invention and include, for example, both naturally-occurring and synthetic (i.e. non-natural) amino acids. Of these, aspartic and glutamic acids are particularly preferred.

Preferred for use in the invention are volatile acids which, if desired, can readily be removed after completion of the reaction to produce the chitosan salt.

Such volatile acids are particularly preferred for use in those embodiments where the chitosan is contacted with the acid in the gaseous or vapour state. By “volatile acid” is meant any acid that may be disassociated from the chitosan salt at a temperature of less than 50° C. and at a pressure of between 30 mbar and ambient pressure. Typically, a volatile acid will have a boiling point less than about 180° C. at ambient pressure. Volatile acids include HCl, acetic acid, trifluoroacetic acid, iso-butyric acid, n-butyric acid, formic acid, propionic acid and pyruvic acid. Preferably, the volatile acid is HCl, acetic acid or propionic acid, particularly preferably acetic acid.

Mixtures of acids may also be used in the method of the invention. Preferred are mixtures of acids with at least one carboxylic acid as hereinbefore described.

The amount of acid used can be varied depending on the desired degree of protonation (and thus solubility) of the final product. The only requirement is that this should be sufficient to protonate enough of the amino groups of the chitosan to yield a soluble product. Appropriate amounts of acid will depend on a number of factors, including the nature of the chitosan starting material (i.e. its degree of deacetylation and thus its content of free amino groups), the nature of the acid (e.g. whether this is mono- or polyprotic), etc., but may be readily determined by those skilled in the art. For example, the amount of acid may be varied from relatively small amounts up to and exceeding essentially stoichiometric amounts. For example, the molar amount of acid for each mole of chitosan may range from as little as 0.4 M. eq. The upper end of the range is less critical, however in general this will not extend beyond 10 moles of acid per mole of free amino groups (D-units) in the chitosan.

Generally, it is preferred that the amount of acid used is sufficient to protonate a major proportion of the amino groups of the chitosan. For example, the amount of acid may range from 40 to 120% of the molar amount of deacetylated (i.e. free amino) units in the chitosan, more preferably from 60 to 110%, yet more preferably from 80 to 100%.

Products containing less than the stoichiometric amount of acid (e.g. as low as 70% of the stoichiometric amount) are also soluble in water and thus within the scope of the invention. Such products may either be obtained by using less acid in their preparation or, alternatively, by removing a larger amount of acid in a post-reaction procedure, e.g. evaporation.

The process of the invention is conducted in a heterogeneous reaction system. As such, the speed and extent of reaction is dependent not only on the particle size of the chitosan but also the degree of hydrogen bonding between individual molecules of chitosan which affect the accessibility of the free amino groups. Other factors affecting the reaction include the nature of the acid, e.g. its strength as measured by its pKa value.

Although the resulting product may be used in the form of a dispersion or finely divided suspension of the chitosan salt in the acid (i.e. without isolation of the chitosan salt), typically the reaction product will be isolated by conventional procedures known in the art. For example, excess acid remaining after conversion of the chitosan into the desired protonated product may be removed by procedures conventional in the art for separating solids from liquids, such as decantation, filtration, centrifugation or any combination thereof. If desired, this may be followed by further drying steps, e.g. evaporation, spray-drying, vacuum drying, air-drying, heating, etc. to produce a substantially dry, solid form. Most conveniently, further drying may be achieved by heating the resulting product to a temperature below the denaturation temperature of the chitosan, e.g. to a temperature in the range of from 0 to 60° C., preferably 5 to 50° C., e.g. to about 40° C. Preferably, the final product is obtained in finely divided form, e.g. as a granulate or particulate which can be readily dissolved in water.

The water content of the chitosan salt before conducting any optional drying step is preferably less than 10% by weight, more preferably less than 3% by weight (based on the total weight of the salt).

In a preferred aspect of the invention, the recovered chitosan salt may be subjected to further heat treatment at an elevated temperature for a period of time. Such heat treatment results in cleavage of glycosidic linkages within the chitosan chains and can therefore be tailored to achieve the desired molecular weight value for the final chitosan product. In general, the chitosan salt may be heat treated at a temperature within the range of from 30 to 50° C., preferably 30 to 40° C., more preferably 35 to 40° C., e.g. about 35° C. The higher the temperature used, the shorter the period needed to achieve the desired degree of cleavage. Typically, heating may be conducted over a period of time of up to 70 hours, preferably up to 60 hours, e.g. about 40 hours. In general, this heat treatment step will be conducted at atmospheric pressure. However, it may also be conducted under a low vacuum.

Chitosan products produced according to the methods herein described form a further aspect of the invention. Typically, these will comprise protonated chitosan in substantially dry, free-flowing form, e.g. in the form of particles having an average particle size in the range from 1 to 10000 μm. As used herein, “free-flowing” means that the product is in particulate form and that it can be poured.

It is preferred that the chitosan salts produced according to the method of the invention should maintain substantially the same particle size as the original chitosan starting material. In this regard, it is particularly preferred that the average particle size of the original material and that of the final chitosan salt should differ by less than 20%, preferably less than 10%, e.g. by less than 5%. The solubility kinetics of the chitosan produced is expected to depend on the molecular weight of the chitosan as well as the viscosity of the solution initially produced. The products produced according to the invention are highly soluble in water. Typically, these will have a water solubility in the range up to 200 g/L, preferably up to 20 g/L.

The chitosan products produced by the methods herein described will contain a residual amount of the free acid. Viewed from a further aspect the invention thus provides a substantially water-soluble chitosan product comprising a protonated chitosan and a residual amount of an acid as herein described. Preferably, the residual amount of acid which will be present in the product will be less than a 100% molar excess of acid to free amine groups in the chitosan starting material, preferably less than a 30% molar excess, more preferably less than a 10% molar excess.

The product produced according to the method of the invention, is a protonated chitosan having a degree of protonation somewhere between no protonation and complete or full protonation. The final composition of the product, i.e. the degree of protonation, is dependent on the relative proportions of chitosan and acid used in the treatment as well as the reaction conditions (time, temperature and pressure). A higher amount of acid and/or a stronger acid will result in a product closer in structure to the fully protonated molecule, whereas the use of a lower amount of acid and/or a weaker acid will result in a product having a much lower level of protonation for given reaction conditions.

The chitosan salt produced by the process of the invention may be used as produced or it may be subject to further chemical or physical modification, e.g. depolymerisation, deacetylation, acetylation, chemical derivatisation, grinding, gel formation, solution formation, fractionation, acid extraction, etc. Typical examples of such techniques are widely described in the scientific and patent literature.

The chitosan salt or modified salt product can then be used in any of the fields of use previously suggested for chitosans, e.g. in the preparation of technological, agricultural, food (including human food and animal feed and feed additives), nutraceutical, pharmaceutical, biomedical, veterinary and cosmetic products. It is particularly suitable however as a pharmaceutical or veterinary composition, food or cosmetic additive.

Thus viewed from a further aspect the invention provides a process for the preparation of a product comprising a water-soluble chitosan salt which process comprises (e.g. consists essentially of) the following steps:
(a) contacting a solid chitosan with at least one protic acid whereby to produce a water-soluble chitosan salt;
(b) optionally recovering said salt; and
(c) formulating said salt with at least one physiologically acceptable carrier or excipient or with at least one foodstuff,
   wherein said chitosan is converted into said salt in step (a) whilst remaining substantially in the solid state.

Where the chitosan salt is intended for use in a pharmaceutical product, this will generally be mixed with the necessary ingredients and then provided in tablet form.

Viewed from a yet further aspect the invention provides the use of a chitosan product of the process of the invention in the preparation of technological, agricultural, food, nutraceutical, pharmaceutical, biomedical, veterinary and cosmetic products.

Viewed from a still further aspect the invention provides a technological, agricultural, food, nutraceutical, pharmaceutical, biomedical, veterinary or cosmetic product containing a chitosan product of the process of the invention.

A further advantage of the chitosan products produced according to the method of the invention relates to their use as foodstuffs. The inventors have found that chitosan substantially in the free amine form with various degrees of acetylation (F_{A}) and molecular weight has an astringent, bitter, seawater-like taste and feeling in the mouth and on the tongue. However, sensory tests performed in relation to the chitosan salts of the invention confirm that these do not have this astringent, bitter and seawater-like taste. This finding increases the potential for the utilisation of such chitosan products in foods and beverages and in food and beverage ingredient systems.

Therefore, viewed from a further aspect, the invention provides a foodstuff or a beverage comprising a chitosan salt as herein described or which is produced by a process as herein described.

The invention will now be further described with reference to the following non-limiting Examples:

### EXAMPLE 1

### Preparation of Chitosan Salts from Concentrated Acetic Acid

Two different chitosans with different degrees of acetylation (F_{A}) in the free amine form were used as starting materials. These were characterized with respect to their water content as well as their intrinsic viscosities (intrinsic viscosities [η] were determined at an ionic strength of 0.1M according to: Draget, K. I., \Thrum, K. M., Moen, E., Gynnild, H. & Smidsrød, O., Biomaterials, (1992), 13, 635-638):

Chitosan 1—F_{A}=0.08; water content=14.7±0.2%; [η]=300 ml/g
Chitosan 2—F_{A}=0.40; water content=17.7±1.2%; [η]=1040 ml/g

Chitosan salts were prepared from Chitosan 1 and Chitosan 2 using concentrated acetic acid as follows:

To 1.000 grams of Chitosan 1 and Chitosan 2 were added 1.00, 2.00, 5.00 and 10.00 mL of concentrated acetic acid (water content: 1%). In each case, the mixture was left for 24 hours at room temperature in a closed 100 mL bottle. Then the samples were dried at 40° C. for 48 hours, and the weight of the samples was determined.

### Results:

Chitosan 1 - F_{A} = 0.08

| mL of added acetic acid | Weight of final product after drying (g) |
|---|---|
| 1.00 | 1.249 |
| 2.00 | 1.259 |
| 5.00 | 1.265 |
| 10.00 | 1.252 |

Chitosan 2 - F_{A} = 0.40

| mL of added acetic acid | Weight of final product after drying (g) |
|---|---|
| 1.00 | 1.220 |
| 2.00 | 1.200 |
| 5.00 | 1.210 |
| 10.00 | 1.219 |

Both chitosan products seemed to reach a constant weight upon drying with increasing amount of acetic acid added. The increase in the weight of the chitosan product decreases with increasing F_{A}. This is to be expected upon formation of a true chitosan acetate salt that can only be formed as counter ions to deacetylated units. The theoretically calculated weights upon forming acetate salts were calculated to be 1.33 g and 1.199 g for the products from Chitosan 1 and Chitosan 2, respectively.

Both chitosan products were found to be readily soluble in water.

### Conclusion:

These experiments show that water-soluble chitosan acetate salts can be formed with chitosans of different F_{A}, and that close to stoichiometric amounts of acetate (in relation to the content of free amino groups in the original chitosan material) are formed upon removing the excess (volatile) acid.

### EXAMPLE 2

### Preparation of Chitosan Salts Using Gaseous Acids

Samples of Chitosan 1 and Chitosan 2 as used in Example 1 (1.000 gram each in the free amine form) were placed in a desiccator at 23° C. where the desiccant was replaced by a Petri dish (15 cm diameter) containing 75 ml of concentrated (37%) hydrochloric acid or glacial acetic acid (99-100%). The air in the desiccator was removed by a water jet pump (to a pressure of 0.1 bar) thus creating an acidic atmosphere in the desiccator from the evaporation of the liquid acid. Chitosan samples were taken out of the desiccator after various times and the weights determined. The solubility of the samples was determined by adding 100 mL of double distilled water and the samples were placed on a shaker (Janke & Kunkel IIKA-VIBRAX-VXR) for 4 hours. The samples were then visually inspected and the solubility classified as insoluble (−), partially soluble with swollen gel particles (+/−), or soluble (+).

### Results:

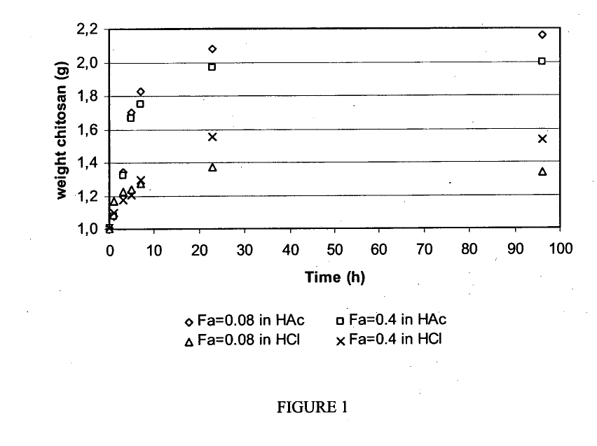

FIG. 1 shows the weight of the two different chitosan samples (F_{A}=0.08 and F_{A}=0.40) as a function of time in 37% hydrochloric acid and glacial acetic acid vapours. The weights of all the chitosan samples increased with time up to 24 hours, with little further increase upon further incubation. It can be seen that the weight increase of chitosans incubated in acetic acid vapour is greater than that of the chitosans incubated in hydrochloric acid vapour, as would be expected from the increased molecular weight of acetic acid (Mr=60.00) compared to that of hydrochloric acid (Mr=36.45).

Table 1 below shows the solubility of the two different chitosan samples after exposure to hydrochloric acid and acetic acid vapours for 1, 2 and 3 hours.

TABLE 1

| | Solubility | | | Solubility | | |
|---|---|---|---|---|---|---|
| | (hours in HCl) | | | (hours in AcOH) | | |
| Chitosan F_{A} | 1 | 2 | 3 | 1 | 2 | 3 |
| 0.08 | − | + | + | − | + | + |
| 0.40 | +/− | + | + | +/− | + | + |

− insoluble
+/− partially soluble with swollen gel particles
+ soluble

All samples incubated for more than 3 hours in acid vapours were soluble (+). The results show that chitosans in the free amine form that are insoluble in double-distilled water can be made soluble in double-distilled water by exposing the chitosans to hydrochloric acid vapour or acetic acid vapour for at least 2 hours at the conditions described in this experiment.

### EXAMPLE 3

### Preparation of Chitosan Salts from Addition of Glacial Acetic Acid Containing Increasing Amounts of Water

1.000 g of chitosan (free amine form) was incubated with 1.00 mL of glacial acetic acid (99-100%) and increasing amounts of water. The glacial acetic acid (1.00 mL) and from 0.10 to 2.00 mL of water were mixed before the total volume of glacial acetic acid and water (1.00 to 3.00 mL) was added to the chitosan sample in 100 mL Pyrex bottles. The mixture was shaken and incubated in the closed bottle (with screw cap) for 24 hours at room temperature, and then dried in the same open bottle (without screw cap) at 40° C. for 48 hours. The weight of the dried chitosan samples was determined and the particle size of the dried samples was estimated. The solubility of the samples was determined as described as in Example 2, except that the solubility was inspected after 24 hours.

### Results:

Chitosans 1 and 2 are as defined in Example 1.
Chitosan 3—F_{A}=0.48; water content: 14.3%; [η]=1210 ml/g

### Chitosan 1—F_{A}=0.08

All samples were soluble in distilled water, except the sample containing the highest water content which was only partially soluble after 24 hours. With increasing water content the dried chitosan samples were increasingly more difficult to dissolve.

The appearance of the chitosan samples that were dried from glacial acetic acid containing increasing amounts of water differed. The chitosan sample dried from glacial acetic acid without added-water appeared as particles of similar size to the untreated chitosan (free amine form), although with a more yellow colour. The chitosan samples dried from acetic acid containing increasing amounts of water contained increasing amounts of larger particles. The chitosan dried from the acetic acid with the highest water content contained a hard brown layer of chitosan that was difficult to remove from the bottle.

### Chitosan 2 —F_{A}=0.40

The appearance of the chitosan samples that were dried from glacial acetic acid containing increasing amounts of water differed. The chitosan sample dried from glacial acetic acid without added water appeared as particles of similar size to the untreated chitosan (free amine form). However, the yellow colour was less obvious as compared to the chitosan with the lower F_{A} (Chitosan 1). The chitosan samples dried from acetic acid containing increasing amounts of water contained increasing amounts of larger particles, similar to that which was observed with the chitosan with the lower F_{A}.

### Chitosan 3—F_{A}=0.48

The appearance of the chitosan samples that were dried from glacial acetic acid containing increasing amounts of water differed. The chitosan sample dried from glacial acetic acid without added water appeared as particles of similar size to the untreated chitosan (free amine form). However, the yellow colour in all dried chitosans was even less obvious as compared to the chitosan with the lower F_{A} (Chitosan 1). The chitosan samples dried from acetic acid containing increasing amounts of water contained increasing amounts of larger particles similar to that which was observed with the chitosan with the lower F_{A} value.

### EXAMPLE 4

### Preparation of Chitosan Salts Using Propionic Acid

Chitosan 1 and Chitosan 2 are as defined in Example 1.

To 1.000 grams of Chitosan 1 and Chitosan 2 were added 1.00, 2.00 and 3.00 mL of concentrated propionic acid. The mixtures were left for 24 hours at room temperature in a closed 100 mL bottle. Then the samples were dried at 40° C. for 240 hours, and the weights of the samples were determined.

### Results:

Chitosan 1 - F_{A} = 0.08

| mL of added propionic acid | Weight after drying (grams) |
|---|---|
| 1.00 | 1.37 |
| 2.00 | 1.41 |
| 3.00 | 1.44 |

Chitosan 2 - F_{A} = 0.40

| mL of added propionic acid | Weight after drying (grams) |
|---|---|
| 1.00 | 1.22 |
| 2.00 | 1.24 |
| 3.00 | 1.29 |

The time to reach an essentially constant weight of the chitosan after addition of propionic acid was much longer as compared with acetic acid.

The appearance of Chitosan 1 was of slightly yellow colour, while this was not the case with Chitosan 2.

Both chitosans are partially soluble in water, containing swollen gel particles. Both chitosans seem to reach a constant weight upon drying with increasing amount of propionic acid added. The increase in the Weight of the chitosan decreases with increasing F_{A}. This is to be expected upon formation of a true chitosan propionate salt that can only be formed as counter ions to deacetylated units. The theoretically calculated weights upon forming propionate salts were calculated to be 1.42 grams and 1.26 grams for, Chitosan 1 and Chitosan 2, respectively.

1. A process for the preparation of a water-soluble chitosan salt which process comprises the following steps:
(a) contacting a solid chitosan with at least one substantially dry, liquid protic acid whereby to produce a water-soluble chitosan salt; and
(b) optionally recovering said salt,
wherein the process is carried out substantially in the absence of any organic solvent.

2. A process as claimed in claim 1 wherein said chitosan is converted into said salt whilst remaining substantially in the solid state.

3. A process as claimed in claim 1 which further comprises heating said salt to a temperature in the range of from 30 to 50° C. for a period of up to 70 hours.

4. A process as claimed in, claim 1 wherein the acid is hydrochloric acid, acetic acid, or propionic acid.

5. A process as claimed in claim 4, wherein the acid is glacial acetic acid.

6. A process for the preparation of a product comprising a water-soluble chitosan salt which process comprises the following steps:
(a) contacting a solid chitosan with at least one substantially dry, liquid protic acid whereby to produce a water-soluble chitosan salt;
(b) optionally recovering said salt; and
(c) formulating said salt with at least one physiologically acceptable carrier or excipient or with at least one foodstuff,
wherein said chitosan is converted into said salt in step (a) whilst remaining substantially in the solid state and wherein the process is carried out substantially in the absence of any organic solvent.

7. A process as claimed in claim 6 which further comprises the step of tableting the resulting product.

8. A process as claimed in claim 6, wherein the solid chitosan of step (a) is produced according to a process comprising swelling particulate chitin with an aqueous solution at a temperature below 30° C. for a period of at least 36 hours, and subsequently reacting the resultant swollen particulate chitin with an alkaline solution at an elevated temperature whereby to cause deacetylation to occur.

9. A process as claimed in claim 1 wherein the solid chitosan of step (a) is a chitosan having a degree of acetylation of 0.45 to 0.7 which on acid hydrolysis with 12M HCl for 9 hours at 40° C. yields a monomer and oligomer mixture which in size exclusion chromatography does not exhibit a pattern of progressively smaller peaks corresponding to monomers, dimers, trimers, tetramers, pentamers and hexamers.

10. A product obtained by a process as claimed in claim 1.

11. A substantially water-soluble chitosan product comprising a protonated chitosan and a residual amount (preferably less than 5% by weight, more preferably less than 2% by weight, e.g. less than 1% by weight of total solids) of a protic acid.

12. A product as claimed in claim 10 having an F_{A} greater than 0.25, preferably from 0.3 to 0.6.

13. A product as claimed in claim 11 having an F_{A} from 0 to 0.3, preferably from 0 to 0.25.

14. A foodstuff or beverage comprising a product as defined in claim 10.
